(19) United States
(12) Patent Application Publication
(10) Pub. No.: US 2020/0390947 A1
(43) Pub. Date: Dec. 17, 2020

(54) BAG FOR CARRYING A PUMP FOR HUMAN BREAST MILK

(71) Applicant: Constance Michelle Simpson, Mobile, AL (US)

(72) Inventor: Constance Michelle Simpson, Mobile, AL (US)

(21) Appl. No.: 16/441,978

(22) Filed: Jun. 14, 2019

(51) Int. Cl. A61M 1/06; A45F 3/04; A45C 11/00; A45C 13/02

(52) U.S. Cl. CPC A61M 1/062; A45F 3/04; A45F 2003/003; A45C 13/02; A45C 11/00

(57) ABSTRACT

A bag for carrying a breast pump, related accessories and personal items is provided, comprising a plurality of interior and exterior pouches for holding various items, and a thermally-insulated interior space for housing a breast pump and collection containers. The bag has at least one handle that facilitates carrying the bag on a shoulder of a human body.

### FIELD OF THE INVENTION

The present invention relates generally to a bag, and in particular, a bag for carrying a pump for extracting human breast milk and multiple accessories related to such a pump.

### BACKGROUND OF THE INVENTION

Breast pumps are a very common and useful device for women who are nursing. They may be used for various purposes, such as for stimulating milk production. Additionally, breast pumps are extremely helpful for women who work away from home or are away from home for any other reason, as they allow to pump their breast milk every few hours, save the milk in a container, and take the milk home with them for their infant at the end of the work day.

Normally, breast pumps are either manually or electrically operated to suction milk from the woman's breast. These devices are used with a set of related equipment or accessories, which can include one or more of a hose, a suction nipple, a breast pad, a collection bottle, a bottle connection, a milk filter, a pump membrane, a handsfree bustier, a nipple shield, a bottle brush, and soap for washing one or more of these accessories. Electrical breast pumps may include additional accessories such as an electrical cord, a case for storing the electrical cord, and the like.

Women who use breasts pumps when away from home are required to carry the breast pump and the associated equipment with them. This can be cumbersome, as women must carry these items together with their own personal items such as a wallet, a cellphone, a tablet, personal hygiene items, makeup, a personal bag or purse, keys, etc. Furthermore, may women do not feel comfortable openly carrying a breast pump and related accessories, since it reveals that she is nursing.

Some containers and bags are known in the art that facilitate carrying the breast pump and related accessories. Many of these containers and bags have various compartments that allow for organizing the contents inside. However, even these can be bulky and indiscreet; they do not resemble a more stylish bag that would better conceal the contents and maintain nursing private. It can still be cumbersome to carry the breast pump and related accessories, and again, it can be obvious in a public or professional setting what is inside. Additionally, current designs can be rather expensive, and a lower-cost option can still be created. Furthermore, most pump bags known in the art are designed to fit a specific breast pump model, and will not fit other breast pumps.

Therefore, there remains a need for a solution that addresses at least one of the above issues. More particularly, there is a need for a carry-on bag or case which allows a woman to conceal and transport a breast pump and related accessories, together with her personal items.

### SUMMARY OF THE INVENTION

The present invention is directed to a bag for carrying and concealing nursing tools including a breast pump and related accessories, together with personal items which the user may need to carry when working or being away from home. The pump bag is configured to keep stored breast milk cool. Multiple storage compartments are provided on the sides of the bag, allowing the user to store a wide variety of accessories and personal items in such a way that the accessories and personal items are organized and within hands reach if needed. The bag facilitates separating dirty or used items from clean, unused items, and also allows to carry backup clothing so that the user can change clothes in the event of a milk leakage onto the user's clothes. The pump bag allows the user to be discreet and stylish while carrying the essential equipment required for breast milk pumping.

Introducing a first embodiment of the invention, the present invention consists of a bag for carrying a breast pump for extracting milk from a female breast. The bag comprises a non-see-through and thermally-insulated bag body having at least one pouch for holding at least one item, and an interior space formed within the bag body and configured to house a breast pump.

In a second aspect, the bag body can include at least one sidewall which is partially surrounded by an inverted U-shaped fastener extending along the top and sides of the sidewall and is pivotably foldable downward and away from a remainder of the bag body to a horizontal position on a surface.

In another aspect, the bag can further include at least one handle carried by the bag body.

In another aspect, the at least one handle can include one or more handles configured to facilitate carrying the bag on a shoulder of a human body.

In another aspect, the at least one handle can include a top handle configured to be gripped by a user's hand.

In another aspect, the bag body can further include a movable, first sidewall provided with at least one inner pouch. The bag can be configured to adopt a transport configuration in which the first sidewall encloses the interior space of the bag body and the at least one inner pouch of the first sidewall faces the interior space of the bag body.

In another aspect, the first sidewall can be pivotably foldable downward and away from a remainder of the bag body to a horizontal position on a surface.

In another aspect, the bag body can further include a movable, second sidewall provided with at least one inner pouch. When the bag is in the transport configuration, the second sidewall can enclose the interior space of the bag body and the at least one inner pouch of the second sidewall can face the interior space of the bag body.

In another aspect, the second sidewall can be pivotably foldable downward and away from a remainder of the bag body to a horizontal position on a surface.

In another aspect, the first and second sidewalls can be arranged on opposite sides of the bag body and spaced apart from one another, with the interior space arranged between the first and second sidewalls when the bag is in the transport configuration.

In another aspect, both the first sidewall and the second sidewall can be pivotably foldable downward and away from a remainder of the bag body to a horizontal position on a surface.

In another aspect, the first and second sidewalls can be partially surrounded by a respective, inverted U-shaped fastener extending along the top and sides of the first and second sidewalls, and can be attached to a remainder of the bag body by a respective, bottom hinge portion.

In another aspect, the bag body can further include a first handle carried by the first sidewall and a second handle carried by the second sidewall.

In another aspect, the first and second handles can be arranged in a side-by-side and spaced-apart relationship with one another when the bag is in the transport configuration.

In another aspect, the first and second handles can be oriented upward when the bag is in the transport configuration.

In another aspect, the bag body can further include an aperture extending through the bag body and communicating the interior space of the bag body with an outside of the bag body.

In another aspect, the bag body can further include a movable cover selectively movable from a closed position in which the movable cover covers the aperture to an open position in which the movable cover does not cover the aperture.

In another aspect, the bag body can further comprise a base having at least one recess facing the interior space and configured to receive a breast pump such that the breast pump is seated in the at least one recess and arranged within the interior space, and concealed from outside the bag by the bag body.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a top front isometric view of a bag for carrying a breast pump for extracting breast milk, in accordance with an illustrative embodiment of the invention, the bag shown closed, and a cellphone shown being inserted into a first exterior pouch of the bag;

FIG. 2 presents a top rear isometric view of the bag of FIG. 1, with a second exterior pouch shown closed, and an aperture shown closed by a flap;

FIG. 3 presents a top rear isometric view of the bag of FIG. 1, with the second exterior pouch shown open and a breast pump electrical cord shown extending through the aperture;

FIG. 4 presents a top rear isometric view of the bag of FIG. 1, with a front sidewall and a rear sidewall shown open and horizontally deployed;

FIG. 5 presents a top front isometric view of the bag in the situation of FIG. 5;

FIG. 6 presents a top rear isometric view of the bag of FIG. 5, with several pump-related accessories being placed in different compartments and ouches of the device; and

FIG. 7 presents a cross-sectional top plan view of the bag of FIG. 1, the cross section taken along section plane 7-7 indicated in FIG. 5.

Like reference numerals refer to like parts throughout the several views of the drawings.

### DETAILED DESCRIPTION

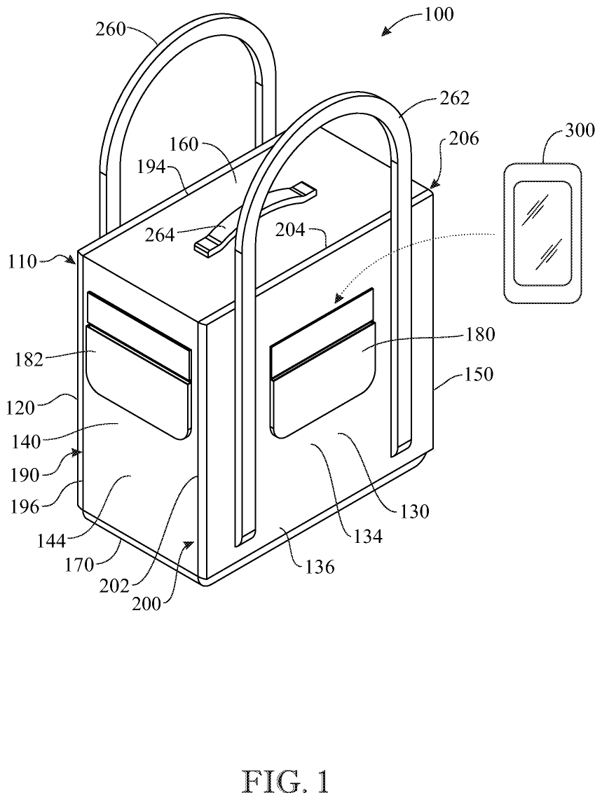

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward a bag for carrying a breast pump, related accessories and personal items.

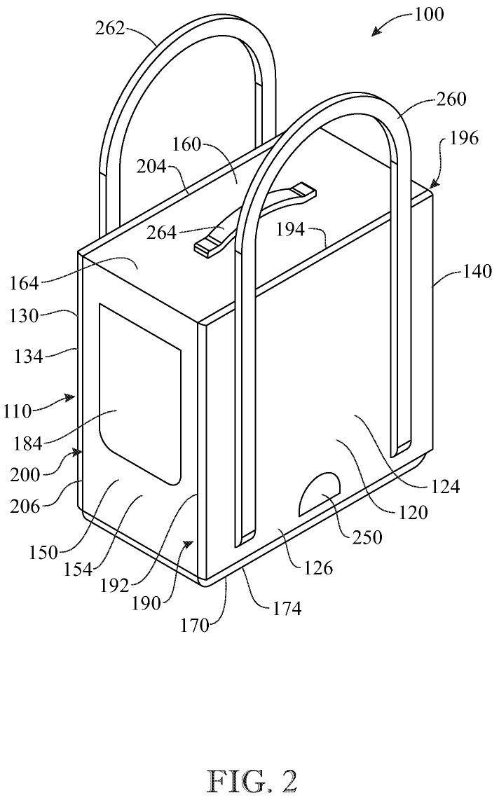

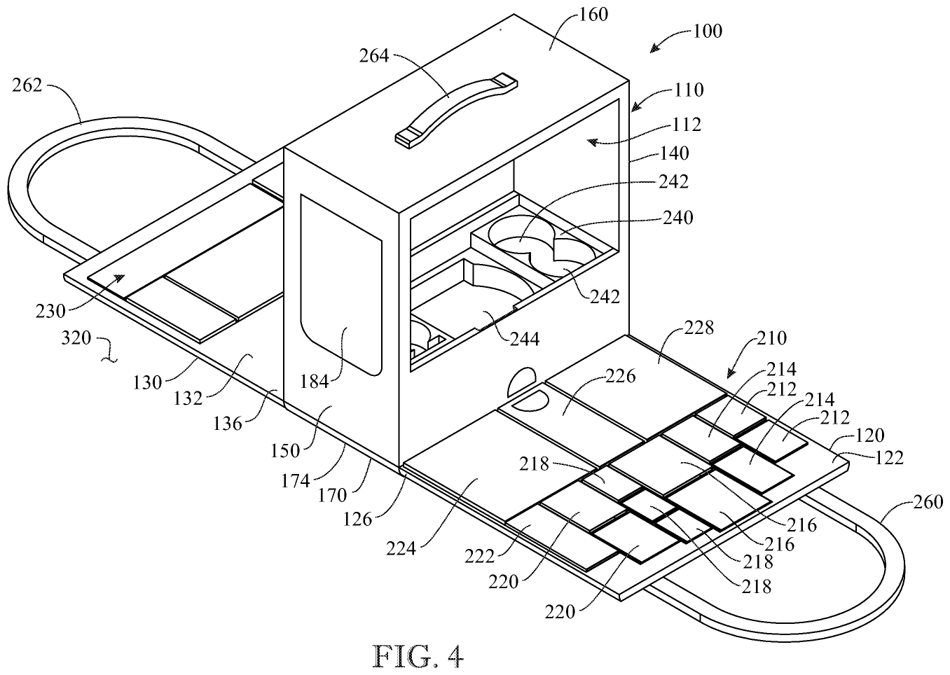

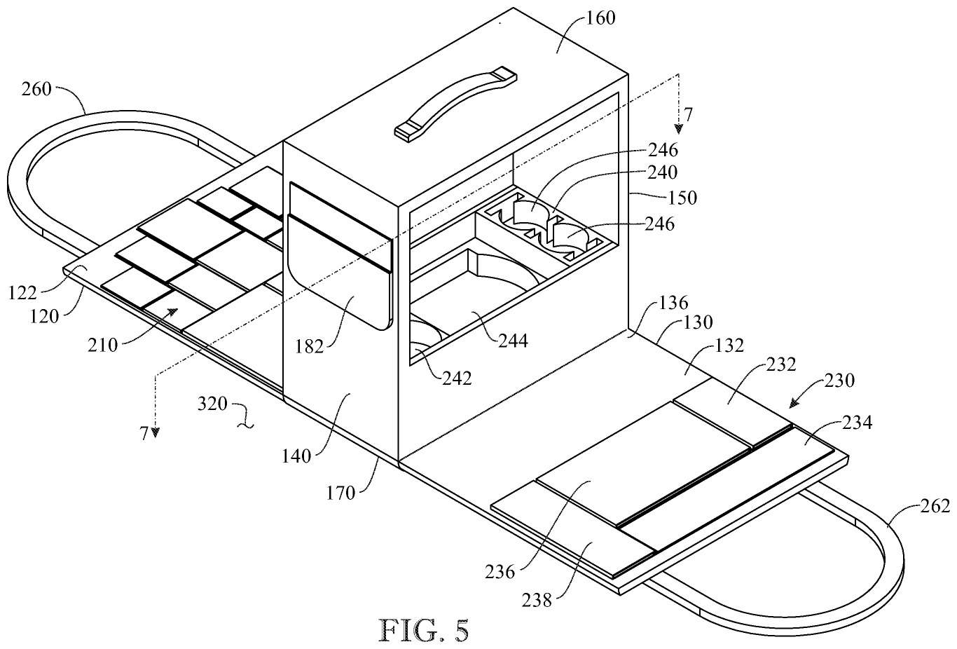

Referring initially to FIGS. 1 and 2, a bag 100 for carrying a breast pump, related accessories and personal items comprises a bag body 110 having a rear sidewall 120, a front sidewall 130, a left sidewall 140, a right sidewall 150, a top wall 160, and a bottom wall 170. Each sidewall has a corresponding inner side and a corresponding outer side. For instance, the rear sidewall 120 has an inner side 122 (FIG. 4) and an outer side 124 (FIG. 2). The front sidewall 130 has an inner side 132 (FIG. 4) and an outer side 134 (FIG. 1). The left sidewall 140 has an inner side 142 (FIG. 4) and an outer side 144 (FIG. 1). The right sidewall 150 has an inner side 142 (FIG. 5) and an outer side 154 (FIG. 2). An interior space 112 is formed inside the bag body 110. The top wall 160 and bottom wall 170 include an outer side 164 and 174, respectively. As will be described in greater detail hereinafter, a breast pump (not shown) can be housed and concealed within the interior space 112. One or more of the sidewalls 120, 130, 140, 150 and walls 160, 170 of the bag body 110, and preferably all sidewalls and walls, are thermally-insulated to maintain the interior space 112 of the bag body 110 cooler than the outside of the bag body 110. Alternatively or additionally, one or more of the sidewalls 120, 130, 140, 150 and walls 160, 170 of the bag body 110, and preferably all sidewalls and walls, are opaque or non-see-through to maintain the interior space 112 of the bag body 110 concealed from the outside observer.

As first illustrated in FIG. 1, a first exterior pouch 180 can be attached to or provided on the outer side 124 of the front sidewall 130 of the bag body 110 for holding at least one item (for instance, a car key or a cellphone 300). In some embodiments, a fastener such as, but not limited to, a zipper, button, snap fastener, magnetic fastener or hook-and-loop fastener, can be included for opening and closing the first exterior pouch 180. A second exterior pouch 182 can be attached to or provided on the outer side 144 of the left sidewall 140 of the bag body 110 for holding at least one personal item such as, but not limited to, a car key or a cellphone 300. The second exterior pouch 182 may comprise a fastener such as, but not limited to, a zipper, button, snap fastener, magnetic fastener or hook-and-loop fastener, for opening and closing the second exterior pouch 182 to respectively allow or prevent access to items stored in the second exterior pouch 182.

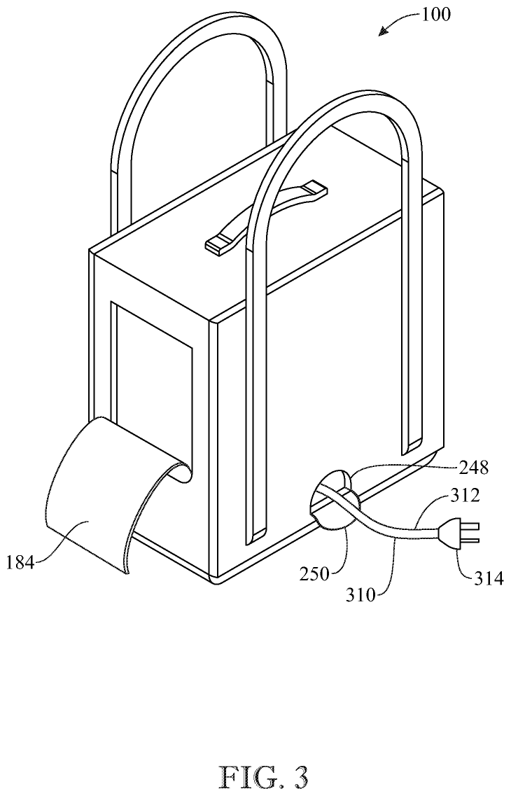

As further shown in FIG. 2, a third exterior pouch 184 can be attached to or provided on the outer side 154 of the right sidewall 150 of the bag body 110, for instance for holding an electrical cord for providing electrical power to the pump. An exemplary electrical cord 310 is shown in FIG. 3 in a usage position in which the cord 310 has been connected to a breast pump (not shown) housed inside the bag body 110 and a distal portion 312 of the cord 310 extends outwardly from the bag body 110 so that an end plug 314 of the cord 310 can be plugged into an electrical socket (not shown) to power the breast pump. When not in use, the cord 310 can be disconnected from the breast pump and stored in the third exterior pouch 184. The third exterior pouch 184 can have a fastener such as, but not limited to, a zipper, button, snap fastener, magnetic fastener or hook-and-loop fastener, for opening and closing the third exterior pouch 184 to respectively allow or prevent access to items stored in the third exterior pouch 184.

As further shown in FIGS. 1 and 2, the bag body 110 depicted herein includes a rear zipper 190 and a front zipper 200 at the rear sidewall 120 and front sidewall 130 of the bag body 110, respectively. The rear zipper 190 has a first vertical portion 192, a transverse or horizontal portion 194 and a second vertical portion 196 forming an inverted U-shaped zipper arrangement. Similarly, the front zipper 200 has a first vertical portion 202, a horizontal portion 204 and a second vertical portion 206 forming an inverted U-shaped zipper arrangement. A bottom portion 126 of the rear sidewall 120 is attached to the remainder of the bag body 110 and provides a hinge. Similarly, a bottom portion 136 of the front sidewall 130 is attached to the remainder of the bag body 110 and provides a hinge. When the rear zipper 190 is unzipped, the rear sidewall 120 of the bag body 110 can pivot outward and downward about the hinge or bottom portion 126 of the rear sidewall 120, and can rest on a surface 320, as shown in FIG. 4. Similarly, when the front zipper 200 is unzipped, the front sidewall 130 of the bag body 110 can pivot outward and downward about the hinge or bottom portion 136 of the front sidewall 130, and can rest on the surface 320, as shown in FIG. 4. While both the rear sidewall 120 and the front sidewall 130 are shown open in FIG. 4, it must be noted that any one of these sidewalls 120, 130 can remain closed and zipped as shown in FIG. 1 while the other one of these sidewalls 120, 130 is open or pivoted downward as shown in FIG. 4.

As first depicted in FIG. 4, the inner side 122 of the rear sidewall 120 comprises a first plurality of pouches 210 for holding a first plurality of items. For instance and without limitation, the first plurality of pouches 210 can include: a pair of pouches 212 for housing, for instance, a respective pair of storage caps; a pair of pouches 214 for housing, for instance, a respective pair of extra nipples; a pair of pouches 216 for storing, for instance, a respective pair of bottle connections; three pouches 218 for storing, for instance, milk filters; two pouches 220 for storing, for instance, pump membranes; a pouch 222 for housing, for instance, a breast pump hose; a pouch 224 for housing, for instance, a handsfree bustier; a pouch 226 for housing, for instance, a bottle brush and soap; and a pouch 228 for housing, for instance, breast pads. Each of the first plurality of pouches 210 can be provided with a fastener such as, but not limited to, a zipper, button, snap fastener, magnetic fastener or hook-and-loop fastener, for opening and closing each of the first plurality of pouches 210.

In turn, as best depicted in FIG. 5, the inner side 132 of the front sidewall 130 of the bag body 110 can include a second plurality of pouches 230 for holding a second plurality of items. These can be a wide range of items. For instance, the second plurality of pouches 230 can include: a pouch 232 for housing, for instance, a snack; a pouch 234 for housing, for instance, a tablet or other electronic device; a pouch 236 for housing, for instance, an extra shirt; and a pouch 238 for housing a water bottle. When the rear and front sidewalls 120 and 130 of the bag body 110 are closed (FIG. 1), the first and second plurality of pouches 210 and 230 are arranged facing one another and spaced apart from one another, separated by the interior space 112 of the bag body 110. When the rear and front sidewalls 120 and 130 are opened (FIGS. 4-6), the first and second plurality of pouches 210 and 230 are pivoted away from one another at opposite sides of the bag body 110. The first plurality of pouches 210 provides a means for storing breast-pumping-related accessories, while the second plurality of pouches 230 provides a separate means for storing personal accessories. Having the first and second plurality of pouches 210 and 230 at opposite sides of the bag body 110 facilitates organizing different-type items (breast-pumping-related accessories on one hand, and personal accessories on the other) on clearly separated and independent areas of the bag 100. For instance, the user may access her personal accessories in the second plurality of pouches 230 without having to open the rear sidewall 120 where the first plurality of pouches 210 is located, and vice versa.

As shown in FIGS. 4 and 5, the bag body 110 further includes a base 240 which can be made of foam, rubber, plastic or other preferably soft materials. The base 240 is elevated relative to the bottom or outer side 174 of the bottom wall 170 and is provided with one or more downwardly-directed recesses or receiving sections, for receiving a breast pump and, optionally, accessories related to the breast pump. For example, the base 240 shown in the drawings includes two generally round, side recesses 242 for receiving nipple shields and/or collection bottles, a central recess 244 for receiving a breast pump, and two opposite, generally round, side recesses 246 for receiving one or more ice packs for cooling the interior space 112 and the items housed within (e.g., collection bottles containing breast milk).

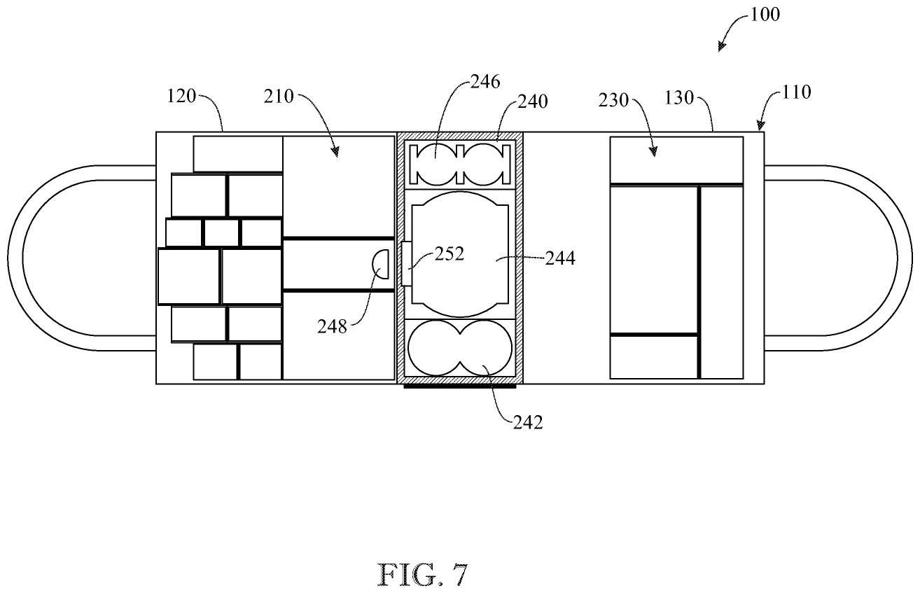

As best shown in FIGS. 3 and 7, an aperture 248 can extend through one of the sidewalls of the bag body 110, communicating the interior space 112 with the outside of the bag body 110 such as to facilitate extending the electrical cord 310 from inside the bag body 110 to outside the bag body 110. For instance, in the present embodiment, the aperture 248 extends through the bottom portion 126 of the rear sidewall 120. A flap 250 on the outer side 124 of the rear sidewall 120 can selectively open or close the aperture 248. The flap 250 can be secured in the closed position by a fastener such as, but not limited to, a zipper, button, snap fastener, magnetic fastener or hook-and-loop fastener. The aperture 248 can be communicated with the central recess 244 via a channel 252 provided on one side of the central recess 244 facing the aperture 248.

As shown in FIGS. 1 and 2, the bag 100 can further include a pair of handles 260, 262 to facilitate holding and carrying the bag 100. More specifically, a rear or first handle 260 is carried by the rear sidewall 120 of the bag body 110 and a front or second handle 262 is carried by the front sidewall 130 of the bag body 110. When the bag 100 is closed, as shown in FIG. 1, the first handle 260 and the second handle 262 are arranged in a spaced-apart relationship with one another and extend upwardly from the bag body 110 forming a respective loop which can fit through a user's arm and onto a user's shoulder, facilitating carrying the bag 100 on the user's shoulder, in addition to carrying the bag 100 with one or two human hands. When the bag 100 is open, as shown in FIGS. 4-7, the first handle 260 is pivoted jointly with the rear sidewall 120 away from the remainder of the bag body 110, and the second handle 262 is pivoted jointly with the front sidewall 130 away from the remainder of the bag body 110. In other words, the handles 260, 262 are pivoted outwardly and away from the internal space 112 of the bag body 110 and thus do not interfere with the user's actions of inserting or removing items from the bag 100.

As further depicted in FIG. 1, the top wall 160 of the bag body 110 can include a central, third handle 264 for holding the bag 100 in a human hand. In some embodiments, as shown, the third handle 264 can be smaller than the first and second handles 260, 262 and can remain substantially between the first and second handles 260, 262 when the bag 100 is in the closed position of FIG. 1.

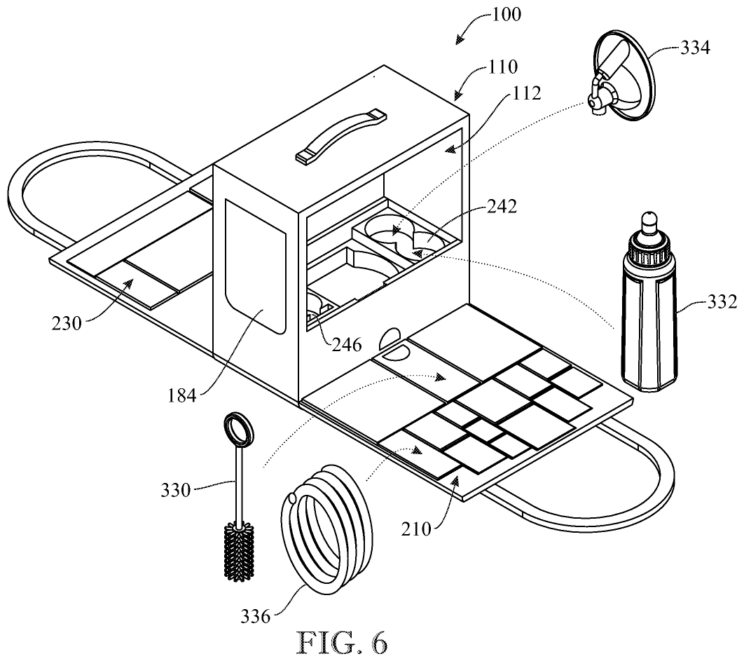

In operation, a user carries the bag 100 by the pair of handles 260, 262 or the central handle 264 just as she would normally carry a purse. The user can store personal items such as a cellphone 300 in compartments such as the first exterior pouch 180, the second exterior pouch 182 or the second plurality of pouches 230. A breast pump (not shown) is seated in the central recess 244 of the base 240. As shown in FIG. 6, accessories such as a bottle-cleaning brush 330, a collection bottle 332 for a woman's breast milk, a nipple shield 334 for covering a nipple of a female human breast, and a breast pump hose 336 can be stored in the side recesses 242, 246 and/or the first plurality of pouches 210. When the user wishes to utilize the breast pump to extract milk from her breast, the user can remove the electrical cord 310 from its storage pouch (e.g., the third exterior pouch 184), plug the cord into the breast pump, extend the cord 310 through the channel 252 and aperture 248 (having previously opened the flap 250) and plug the end plug 314 of the cord 310 into an electrical socket (not shown). In an alternative application, the electrical cord 310 may be non-disconnectably connected to the breast pump; in this event, the user may extend the electrical cord 310 (which can be stored together with the breast pump in the interior space 112 when not in use) through the channel 252 and aperture 248 and equally plug the end plug 314 of the cord 310 into an electrical socket.

In summary, the bag or carrier disclosed herein allows for convenient and organized transporting of nursing tools and personal items in a single container. The container includes multiple storage compartments and an interior space that is preferably thermally-insulated and concealed.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A bag for carrying a breast pump, comprising:
a non-see-through and thermally-insulated bag body having at least one pouch for holding at least one item; and
an interior space formed within the bag body and configured to house a breast pump.

2. The bag of claim 1, wherein the bag body comprises at least one sidewall which is partially surrounded by an inverted U-shaped fastener extending along the top and sides of the sidewall and is pivotably foldable downward and away from a remainder of the bag body to a horizontal position on a surface.

3. The bag of claim 1, further comprising at least one handle carried by the bag body.

4. The bag of claim 3, wherein the at least one handle comprises one or more handles configured to facilitate carrying the bag on a shoulder of a human body.

5. The bag of claim 3, wherein the at least one handle comprises a top handle configured to be gripped by a user's hand.

6. The bag of claim 1, wherein the bag body further comprises a movable, first sidewall provided with at least one inner pouch, and further wherein the bag is configured to adopt a transport configuration in which the first sidewall encloses the interior space of the bag body and the at least one inner pouch of the first sidewall faces the interior space of the bag body.

7. The bag of claim 6, wherein the first sidewall is pivotably foldable downward and away from a remainder of the bag body to a horizontal position on a surface.

8. The bag of claim 6, wherein the bag body further comprises a movable, second sidewall provided with at least one inner pouch, and further wherein, when the bag is in the transport configuration, the second sidewall encloses the interior space of the bag body and the at least one inner pouch of the second sidewall faces the interior space of the bag body.

9. The bag of claim 8, wherein the second sidewall is pivotably foldable downward and away from a remainder of the bag body to a horizontal position on a surface.

10. The bag of claim 8, wherein the first and second sidewalls are arranged on opposite sides of the bag body and spaced apart from one another, with the interior space arranged between the first and second sidewalls when the bag is in the transport configuration.

11. The bag of claim 8, wherein the first and second sidewalls are pivotably foldable downward and away from a remainder of the bag body to a horizontal position on a surface.

12. The bag of claim 11, wherein the first and second sidewalls are partially surrounded by a respective, inverted U-shaped fastener extending along the top and sides of the first and second sidewalls, and are attached to a remainder of the bag body by a respective, bottom hinge portion.

13. The bag of claim 8, wherein the bag body further comprises a first handle carried by the first sidewall and a second handle carried by the second sidewall.

14. The bag of claim 13, wherein the first and second handles are arranged in a side-by-side and spaced-apart relationship with one another when the bag is in the transport configuration.

15. The bag of claim 14, wherein the first and second handles are oriented upward when the bag is in the transport configuration.

16. The bag of claim 1, wherein the bag body further comprises an aperture extending through the bag body and communicating the interior space of the bag body with an outside of the bag body.

17. The bag of claim 16, wherein the bag body further comprises a movable cover selectively movable from a closed position in which the movable cover covers the aperture to an open position in which the movable cover does not cover the aperture.

18. The bag of claim 1, wherein the bag body further comprises a base having at least one recess facing the interior space and configured to receive a breast pump such that the breast pump is seated in the at least one recess and arranged within the interior space, and concealed from outside the bag by the bag body.

19. A bag for carrying a breast pump, comprising:
a non-see-through and thermally-insulated bag body comprising:
an interior space formed within the bag body and configured to house a breast pump;
a movable, first sidewall provided with at least one inner pouch; and
a movable, second sidewall provided with at least one inner pouch; wherein
the bag is configured to adopt a transport configuration in which the first and second sidewalls enclose the interior space of the bag body and the at least one inner pouch of the first and the at least one inner pouch of the second sidewall face the interior space of the bag body; and further wherein
the first and second sidewalls are pivotably foldable downward and away from a remainder of the bag body to a horizontal position on a surface.

20. A bag for carrying a breast pump, comprising:
a non-see-through and thermally-insulated bag body comprising:
an interior space formed within the bag body and configured to house a breast pump;
a movable, first sidewall provided with at least one inner pouch; and
a movable, second sidewall provided with at least one inner pouch; wherein
the first and second sidewalls are arranged on opposite sides of the bag body and spaced apart from one another; wherein
the bag is configured to adopt a transport configuration in which the interior space is arranged between and enclosed by the first and second sidewalls, and further in which the at least one inner pouch of the first and the at least one inner pouch of the second sidewall face the interior space of the bag body; and wherein
the first and second sidewalls are partially surrounded by a respective, inverted U-shaped fastener extending along the top and sides of the first and second sidewalls, and are attached to a remainder of the bag body by a respective, bottom hinge portion, and further wherein the first and second sidewalls are pivotably foldable downward and away from a remainder of the bag body to a horizontal position on a surface.
